(19) United States
(12) Patent Application Publication
(10) Pub. No.: US 2007/0082063 A1
(43) Pub. Date: Apr. 12, 2007

(54) Risk assessment and correction of membrane damage of the upper GI tract

(76) Inventors: Douglas Bibus, Austin, MN (US); Stephen Phinney, Elk Grove, CA (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 11/247,516

(22) Filed: Oct. 11, 2005

(51) Int. Cl. A61K 31/22; A61K 31/202; A61K 31/355; A61K 33/34; A61K 33/04; A61K 33/32

(52) U.S. Cl. 424/638; 424/641; 424/702; 514/458; 514/547; 514/560

(57) ABSTRACT

The present invention is directed to a variety of embodiments. For example, in at least one embodiment the invention is directed to formulations and methods for diagnosis of risk and for treatment to reduce this risk of upper gastrointestinal bleeding from erosions and ulcers. The compositions contain arachidonic acid, such as, but not limited to, arachidonate, 20:4n-6, and may include one or more forms of tocopherol, such as, but not limited to, alpha-, beta-, gamma-, delta- or mixtures thereof, anti-inflammatory lipids, such as, but not limited to, fish oil, DHA, gamma-linolenic acid, and may also include trace minerals required for superoxide dismutase production, such as, but not limited to, copper, zinc, and manganese.

### BACKGROUND OF THE INVENTION

1. Field of the Invention

This invention relates to the testing, risk assessment and treatment for damage to the upper gastrointestinal (GI) tract. Most particularly to food and dietary supplement compositions containing arachidonic acid and to uses thereof in the correction and prevention of membrane damage of the upper GI tract.

2. Description of the Related Art

Upper gastrointestinal bleeding from erosions and ulcers is a common malady and affect both humans and animals. Ulcers are sores that form in the stomach or the upper part of the small intestine, called the duodenum. Most peptic ulcers are caused by a particular bacterial infection in the stomach and upper intestine, by certain medications, or by smoking. Injury of the gastric mucosal lining and weakening of the mucosal defenses are also responsible for gastric ulcers. Excess secretion of hydrochloric acid, genetic predisposition, increasing age, and psychological stress are important contributing factors in the formation and worsening of gastric and duodenal ulcers.

Most ulcers occur in people infected with H. pylori. Typically, when H. pylori bacteria do cause ulcers, the ulcers develop when bacteria weaken the protective coating of the stomach and upper small intestine. Acid in the stomach then gets through to the sensitive tissues lining the digestive system underneath. Acid and bacteria directly irritate this lining resulting in sores, or ulcers.

Although H. pylori contribute to most cases of peptic ulcers, these ulcers can happen for other reasons. Sometimes people regularly take pain relievers that fight inflammation in the body. These medications, known as nonsteroidal anti-inflammatory drugs (NSAIDs), are used to treat certain long-term painful conditions like arthritis. If these medicines are taken in high daily doses over a long period of time, they can cause ulcers in some of the people who use them. The mechanism through which NSAIDs promote ulcer formation is by preventing the production of protective prostaglandins from their precursor arachidonic acid (ARA).

Smoking is also associated with peptic ulcers. Smoking increases a person's risk of getting an ulcer because the nicotine in cigarettes causes the stomach to produce more acid. The consumption of excess alcohol can also increase a person's risk of ulcers because over time alcohol can reduce the resistance of the lining of the stomach and intestines. Specifically, the chronic consumption of excess ethanol blocks the body's production of arachidonic acid (ARA, 20:4ω6), a cytoprotective fatty acid (ref Nakamura M T et al, J Clin Invest 93:450-454, 1994).

In certain circumstances stress can help cause ulcers. But this usually only happens in situations when illness involving severe emotional or physical stress is involved. Ulcers occur because of uncontrolled increased acid production in the stomach and changes in a person's immune system. With any illness where the body's ability to heal is challenged, there is a risk for developing ulcers. The incidence of ulcers is also increased with aging, which may be related to reduced healing.

Stomach pain is the most common symptom of an ulcer. Other symptoms of ulcers can include: loss of appetite; sudden, sharp stomach pains; nausea; frequent burping; weight loss; vomiting; and bloody or blackish bowel movements.

One test to check for an ulcer is called an upper gastrointestinal (GI) series. This is a type of X-ray of the stomach, duodenum, and esophagus. A person drinks liquid containing barium while getting an X-ray, and if he or she has an ulcer, it should be outlined on the X-ray. Another common procedure to look for an ulcer is called an endoscopy. This study involves direct examination of the lining of the stomach and small bowel using a flexible fiber-optic endoscope. Tissue can be removed during an endoscopy and then tested for H. pylori bacteria. A doctor can also do a blood test for H. pylori bacterial antibodies. This may be important if an ulcer is found in the upper GI series or is suspected before the endoscopy.

In order to protect against gastrointestinal damage, the upper GI tract has, among other defenses, within its lining membrane highly unsaturated fatty acids (HUFA). The most prevalent membrane HUFA in the GI tract is ARA, which is used to produce protective compounds called prostaglandins.

When the body is under stress due to stressful situations or intense physical exercise, the body's intake of oxygen increases. Within the body's mitochondria, oxidative phosphorylation occurs and produces reactive oxygen species (ROS) as a by-product. This ROS generation represents about 3% of total body oxygen consumption. Because oxygen consumption is increased with exercise, stress, or inflammation, ROS generation also increases.

Cells have multiple levels of defense around mitochondria to contain ROS resulting from oxidative metabolism. The primary targets of ROS that escape from this containment are HUFA, which are irreversibly degraded by an interaction with ROS. Membrane HUFA in mammals (including dogs and humans) are predominantly from two classes of essential fatty acids. They are arachidonic acid, an omega-6 fat, and abbreviated 20:4ω6, and docosahexaenoic acid (DHA), an omega-3 fat, and abbreviated 22:6ω3.

Arachidonic acid (ARA) is a long chain polyunsaturated fatty acid (PUFA) of the omega-6 class (5, 8, 11, 14-eicosatetraenoic acid, i.e., 20:4). ARA is the most abundant C₂₀ PUFA in the human body. It is particularly prevalent in organ, muscle and blood tissues, serving a major role as a structural lipid associated predominantly with phospholipids in blood, liver, muscle and other major organ systems. In addition to its primary role as a structural lipid, ARA also is the direct precursor for a number of circulating eicosanoids such as prostaglandin E₂ (PGE₂), prostacyclin I₂ (PGI₂), thromboxane A₂ (Tₓ A₂), and leukotrienes B₄ (LTB₄) and C₄ (LTC₄). These eicosanoids exhibit regulatory effects on lipoprotein metabolism, blood rheology, vascular tone, leukocyte function and platelet activation. A more detailed discussion of ARA and its uses may be found in U.S. Pat. No. 5,658,767, which is incorporated herein by reference in its entirety.

In resting cells, arachidonic acid is stored within the cell membrane, esterified to glycerol in phospholipids. A receptor-dependent event, requiring a transducing G protein, initiates phospholipid hydrolysis and releases the fatty acid into the intracellular medium. Three enzymes may mediate this deacylation reaction: phospholipase A2 (PLA2), phospholipase C (PLC), and phospholipase D (PLD), which differ in their sites of attack on the phospholipid backbone. PLA2 catalyzes the hydrolysis of phospholipids at the sn (stereospecific numbering)-2 position. Therefore, this enzyme can release arachidonate in a single-step reaction. By contrast, PLC and PLD do not release free arachidonic acid directly. Rather, they generate lipid products containing arachidonate (diacylglycerol and phosphatidic acid, respectively), which can be released subsequently by diacylglycerol- and monoacylglycerol-lipases.

Once released, free arachidonate has four possible fates: reincorporation into phospholipids, diffusion outside the cell, and metabolism, and non-enzymatic oxidative destruction. Metabolism is carried out by three distinct enzyme pathways expressed in a variety of cells: cyclooxygenase, lipoxygenases, and cytochrome P450. Several products of these pathways act within cells to modulate the activities of ion channels, protein kinases, ion pumps, and receptor-mediated uptake systems. The newly formed eicosanoids may also exit the cell of origin and act at a distance, by binding to G-protein-coupled receptors present on nearby cells. Finally, the actions of the eicosanoids may be terminated by diffusion, uptake into phospholipids, or enzymatic degradation. A further discussion of arachidonate and arachidonic acid can be found in “Arachidonic Acid” written by Daniele Piomelli (Professor of Pharmacology; 360 Med Surge II; University of California, Irvine; Irvine, Calif. 92697-4625), which may found at http://www.acnp.org/g4/GN401000059/CH059.html.

The fourth possible fate of ARA is attack by ROS (activated [or free-radical] oxygen compounds). This process occurs spontaneously without the involvement of enzymatic control. It is driven by the rate of ROS escape from mitochondrial containment, and can attack ARA and other HUFA both in membrane phospholipids and in the free form within intra- and extra-cellular fluids.

If there were no cellular mechanisms to contain and remove ROS, an adult human at rest would produce enough ROS in a day to destroy about 100 grams of membrane HUFA, which is a major fraction of the adult human body's total HUFA content. To prevent rampant destruction of this essential membrane material, cells have multiple “layers” of containment defenses against ROS produced as by-products of oxidative metabolism. These containment defenses include manganese superoxide dismutase (SOD) localized within mitochondria, and copper-zinc SOD localized in the cellular fluid surrounding mitochondria, glutathione, and various tocopherols (alpha-T in the membrane and gamma-T [and its metabolite gamma-CEHC] in the cellular fluid phase).

A reduction in membrane protection against ROS has been noted in a number of situations. Women with gestational diabetes, which is a condition associated with increased ROS, have reduced red blood cell membrane arachidonic acid [ref: Lin H, Diabetologia. 2004; 47:75-81]. In a genetic obesity model with increased inflammation and ROS, obese Zucker rats which were exercised for nine weeks had reduced arachidonate in skeletal muscle and heart, whereas the lean Zucker genotype were less inflamed and showed increased 20:4ω6 in these muscles after exercise [ref: Ayre K J. J Appl Physiol 1998; 85:1898-1902].

Arachidonate plays a pivotal role in protection of the gastric mucosa from ulcer formation, serving as an obligate substrate for prostaglandin synthesis. Aspirin and other NSAIDs have been shown to promote ulcers by blocking prostaglandin formation from ARA, which highlights the central role that these arachidonate metabolites play in mucosal defense against ulcer formation. In further support of this fact, fish oil (containing omega-3 HUFA) has also been found to displace membrane arachidonate and promote stress ulcers in rats [ref: Olafsson SO. Lipids 2000; 35:601-5].

Arachidonate has been given to humans or animals with normal membrane arachidonate content, however this had little effect on serum or tissue arachidonate content (ref: Nelson G J et al. Lipids 32:427-434, 1997). However, an intermediate precursor (GLA) of arachidonic acid given to animals with reduced membrane arachidonate can significantly increase membrane arachidonate content (ref: Phinney S D et al, Metabolism 42:1127-1140, 1993).

Accordingly, there remains a need for an economical, commercially feasible method of evaluating the risk of and the treatment of upper gastrointestinal bleeding from erosions and ulcers. It is an object of the present invention to satisfy that need.

All US patents, applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

The present invention is directed to a variety of embodiments involving formulations and methods for diagnosis of risk and for treatment to reduce this risk of upper gastrointestinal bleeding from erosions and ulcers, which is referred to herein as burnt membrane syndrome (BMS). The compositions all contain arachidonic acid, such as, but not limited to, arachidonate, and may include one or more forms of tocopherol, such as, but not limited to, gamma-, delta-tocopherol or mixtures thereof, anti-inflammatory lipids, such as, but not limited to, fish oil, DHA, gamma-linolenic acid, and may also include trace minerals required for superoxide dismutase production, such as, but not limited to, copper, zinc, and manganese.

In at least one embodiment, the present invention is directed to a method of repleting membrane ARA in an animal or human subject at risk for developing upper GI mucosal damage, which includes, but is not limited to, gastric erosions, gastric ulcers and duodenal ulcers. The subjects to be assessed may be at risk for a number of situations, including, but not limited to, prolonged intense exercise, severe emotional stress, chronic infectious (septic) stress, smoking tobacco products, ethanol (alcohol) use, and advanced age (about 6 years and older). These situations are at risk of being associated with a reduction in membrane ARA.

In at least one embodiment, the present invention is directed to a method of detecting and/or assessing risk for BMS, wherein the risk of upper GI mucosal damage is due, but not limited to, prolonged intense exercise, severe emotional stress, chronic infectious (septic) stress, ethanol use, and smoking tobacco products.

In at least one embodiment, the present invention is directed to treating subjects who have BMS or subjects who are at risk of having BMS by feeding them dietary ARA. The dietary ARA may be supplemented with an anti-inflammatory tocopherol, such as gamma-tocopherol, or delta-tocopherol.

In at least one embodiment, the present invention is directed to companion animal foods formulated to include nutritional supplements appropriate for the specific type of companion animal, such as, but not limited to, cat food, dog food and horse food, the animal food further containing ARA. The dietary ARA may be supplemented with a tocopherol, such as gamma-tocopherol, or delta-tocopherol.

In the present methods, the presence of BMS or the risk thereof is determined through analysis of cheek mucosal cells (buccal cells), gastric or small bowel mucosal cells, red or white blood cells, serum or plasma phospholipids, or serum or plasma total lipids. This is achieved by analyzing the levels of certain fatty acids, most notably ARA.

In at least one embodiment, the present invention is directed to a test kit for extracting mucosal cells from a subject and shipping the samples cells to a test facility for ARA analysis to determine the presence of BMS.

These and other embodiments, which characterize the invention, are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described embodiments of the invention.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

The present invention is directed to a variety of embodiments involving formulations and methods for diagnosis of risk and for treatment to reduce this risk of upper gastrointestinal bleeding from erosions and ulcers, which is referred to herein as burnt membrane syndrome (BMS). The compositions all contain arachidonic acid, such as, but not limited to, arachidonate, and may include one or more forms of tocopherol, such as, but not limited to, gamma-, delta- or mixtures thereof, anti-inflammatory lipids, such as, but not limited to, fish oil, DHA, gamma-linolenic acid, and may also include trace minerals required for superoxide dismutase production, such as, but not limited to, copper, zinc, and manganese.

GI damage may result from excessive and prolonged exercise. GI bleeding from upper GI erosions is common in racing dogs and in human marathon runners. In developing the present invention, a fatty acid test was run on sled dogs to determine BMS occurrences. A base line level of ARA was established from the dogs at rest and samples from post race dogs were then tested for BMS. The test identified levels of omega 3 and omega 6 fatty HUFA, with particular interest paid to levels of arachidonic acid (20:4ω6 or ARA). Other fatty acids targeted by the assay (because they are are susceptible to BMS) are docosahexaenoic aicd (DHA), eicosapentaenoic acid (EPA), docosapetaenoic acid (DPA -omega 6 and 3) and a host of related compounds. These fatty acids have double bond confugurations that make them susceptible to destruction by ROS and thus useful biomarkers of BMS.

Dogs have particularly high levels of arachidonic acid. With levels around 20-30% in plasma phospholipids and 18-25% in red cell membranes, dogs have over two times that of an average human (12% in both tissues). Reductions in these species-specific levels of ARA, DHA, EPA and related compounds are biomarkers of BMS. In addition to functioning as a biomarker of BMS, a reduced level of ARA is also an important contributing factor to the generation of gastric ulcers in dogs and humans, as this unique fatty acid is the precursor for certain compounds called eicosanoids, which protect the gut lining from acid attack. It has been found that the decreased amount of ARA, which is the substrate of the COX (Cyclooxygenase) enzyme which converts the ARA into the protective compounds of the protective lining of the GI, caused by BMS is the contributing or causal factor for developing gastric lesions/ulceration. Thus, the testing of ARA levels serves as a biomarker for BMS and ulcer risk, where BMS or risk thereof is indicated by a reduction in the species-specific level of ARA.

A Burned Membrane Syndrome study was performed on a number of sled dogs in the development of the present invention. This study was undertaken to evaluate the hypothesis that a prolonged period of intense exercise has a negative impact on the essential fatty acid content of cell membranes due to excessive oxidative stress. This hypothesis is relevant to the health of both dogs and humans because it has been observed that dogs running prolonged race events and humans running a competitive marathon have a high prevalence of gastric erosions immediately following the event. In addition, chronic low grade gastrointestinal blood loss is a common observation in humans during intense endurance training.

The present invention links the destruction of a particular membrane essential fatty acid (arachidonic acid, or ARA) by exercise-induced reactive oxygen species to reduced cyto-protection of the gastric mucosa. ARA is the substrate for gastric eicosanoid synthesis, and this class of compounds protects the mucosa of the stomach and small bowel from acid erosion and ulcer formation. Aspirin and other non-steroidal anti-inflammatory drugs cause gastric ulcers by blocking the production of these protective eicosanoids. However up to the present, there has been no observation linking GI tract membrane ARA destruction with intense exercise.

To perform an initial test of this hypothesis, samples were collected from 3 dog teams, 2 teams running the Iditarod (teams #1 and #2), and one participating in the Yukon Quest (team #3). Red blood cell samples were obtained from teams #1 and #3 and cheek mucosal swabs were obtained from at least some of the animals from all three teams.

Both sets of samples were subjected to fatty acid extraction, thin layer chromatographic separation, and gas chromatographic analysis performed by Lipid Technologies LLC (Dr. Doug Bibus, Austin, Minn.). Fatty acids were identified against known standards, and the composition of the phospholipid fractions were expressed as weight percent.

Red cells from 6 dogs per team from teams #1 and #3 obtained before and after the race demonstrate highly variable responses between the two teams, but consistent data within the teams. Whereas red cells from team #3 showed no significant reductions in either omega-3 or omega-6 essential fatty acids (including ARA) across the race, red cells from team #1 showed marked reductions in membrane omega-3 fatty acids, and variable reductions in arachidonic acid.

In contradistinction to the variable responses of the red cells to prolonged exercise, cheek cell analysis from all three teams revealed moderate to marked reductions in ARA, with commensurate increases in the saturated fatty acid 16:0. These data are shown in the table below. Note also that teams #1 and #2 showed marked reductions in the omega-3 fatty acids EPA and DHA, whereas the membrane proportions of both of these omega-3 fatty acids rose in the buccal cells from team #3.

| | Team #1 (n = 5) | | Team #2 (n = 3) | | Team #3 (n = 4) | |
|---|---|---|---|---|---|---|
| | Before | After | Before | After | Before | After |
| 16:0 | 15.41 | 22.13 | 15.89 | 17.97 | 18.09 | 21.39 |
| ARA | 7.25 | 2.42 | 6.44 | 3.85 | 7.27 | 5.74 |
| EPA | 0.86 | 0.34 | 2.13 | 0.62 | 0.79 | 1.09 |
| DHA | 0.36 | 0.17 | 1.40 | 0.44 | 0.73 | 1.27 |

Red blood cells were selected as the reference standard for analysis in this study because they are readily obtainable, physiologically important, and are less subject to day-to-day dietary variation than serum lipids. The cheek cell assay was included to assess a novel method to obtain a non-invasive sample of mucosal cells from the GI tract. In addition, given the more rapid turnover of buccal cells and their anatomical location in the GI tract, they have the potential to better reflect the effects of a 10-day period of oxidative stress on membrane fatty acids than do red cells.

The data from this initial study to determine if burned membrane syndrome occurs in racing sled dogs supports the following conclusions:
1) Buccal cell membranes are more responsive to a 10-day period of intense exercise stress than red cells.
2) The changes seen in buccal cell membranes in teams #1 and #2 (and to some extent from team #3) were both dramatic and confirmatory for our proposed mechanism for the causation of exercise-induced mucosal damage and GI bleeding.
3) The relative consistency within teams and variability between teams suggests that dietary factors before and during a 10-day race can influence membrane composition. Specifically it appears that team #2 was fed a considerable amount of EPA and DHA (i.e., fish or seal) before the race, and that team #3 was fed fish or seal during the race.
4) Diet notwithstanding, all teams showed decreases in buccal cell ARA, indicating that this tissue is a sensitive and effective assay for burned membrane syndrome.
5) This study represents an important first step in establishing burned membrane syndrome as the mechanism through which prolonged exercise leads to GI mucosal damage and bleeding. And as noted in conclusion #3 above, it appears that dietary intervention, particularly during a race, may influence mucosal cell fatty acid composition and thus GI mucosal cyto-protection.

In humans, levels of ARA are fairly tightly conserved in specific tissue lipids. Control is exerted by regulation of ARA production from the essential precursor 18:2ω6, and by selective uptake of ARA in the membrane phospholipid fraction. With the exception of extreme dietary deficiency of the 18:2ω6 precursor (which is rare), production of ARA does not normally limit cell membrane ARA level. As such, a reduced level or lowering of ARA is indicative of excess oxidative stress (BMS). A 20% reduction from tissue-specific normal values is the threshold for mild BMS, and a >50% reduction below these values indicates severe BMS.

There are several tissues and fluids that may be targeted for testing to get levels of fatty acids or the biomarker for BMS. They include, but are not limited to, whole blood, plasma, serum, red blood cells, buccal cells and saliva containing cell fragments and fatty acids. Because the level of ARA in cell membranes is tissue specific (that is, it can differ between organs or organelle's within one individual), the buccal cell membrane is better suited for diagnosing ulcer risk as the buccal mucosa is part of the GI tract. Buccal cells also provide a more rapid or acute window of BMS as their life cycle that entails lipid remodeling is on the order of days and not months in the case of the RBC. Buccal cells also offer a measurement of an actual membrane whereas plasma or serum levels only reflect transport or dietary influenced levels.

After the BMS has been positively identified, the subject is treated with dosage(s) of ARA in the range of 2-10 mg/kg for humans and 5-20 mg/kg for dogs.

A goal in treating a human (or a dog) with burned membrane syndrome (BMS) is to correct the low level of arachidonate in their membrane phospholipids. While this involves giving the subject some dietary arachidonate, efficiently correcting BMS also requires additional dietary supplements to counter the underlying causes of the initial arachidonate depletion (usually due to increased oxidative stress, inflammation, and excess ROS production). This involves reducing the rate at which ROS escape from mitochondria, which is achieved by some combination of the following: 1) increasing anti-oxidant intake, 2) optimizing intake of trace minerals required for superoxide dismutase (SOD) function (copper, zinc, and manganese), and 3) reducing the animal or human's systemic level of inflammation. Thus, in at least one embodiment of the invention, effective treatment of BMS uses a formulation involving arachidonate, one or more anti-oxidant nutrients such as a tocopherol that function to preserve arachdionate and other HUFA within the body, trace minerals to optimize SOD function, and one or more anti-inflammatory nutrients such as omega-3 fats or gamma-tocopherol to suppress inflammation as an cause of oxidative stress. The components of this formulation thus work in synergy to efficiently correct the membrane deficiency in arachidonate better than can be done using arachidonate alone.

In one embodiment, the dosage of arachidonate for an adult human is 200 mg per day. This may be combined with 200 mg per day of gamma-tocopherol, which is obtained by adding 300 mg of mixed tocopherols derived from soy or corn oil or the like (both are 60-65% gamma-tocopherol). In some embodiments, additional components of the formulation include 200 mg per day of docosahexaenoic acid (DHA, 22:6n-3) provided either as fungal oil or DHA-rich fish oil, 2 mg per day of elemental copper; 15 mg per day of elemental zinc, and 2 mg per day of elemental manganese.

In another embodiment, the dosage range for children and adult humans for ARA is 2-10 mg/kg of body weight per day, that for gamma-tocopherol (from mixed soy or corn tocopherols) is 0.5-5 mg/kg/day, and that for DHA (from fungal oil or fish-oil) is 2-10 mg/kg/day. In another embodiment, the dosage range for dogs for ARA is 5-20 mg/kg/day, that for gamma-tocopherol is 0.5-10 mg/kg/day, and that for DHA (from fungal oil or fish-oil) is 2-20 mg/kg/day.

As an example, a formulation to prevent or treat BMS in a 70 kg human would consist of a mixture of 500 mg of fungal oil containing 40-45% ARA, 500 mg of fungal oil containing 40-45% DHA, and 150 mg of soy mixed tocopherols proving 100 mg of gamma-tocopherol. This 1150 mg of mixture would be divided between 2 soft-gel tablets each with a fill-weight of 575 mg. If trace minerals were included in the formulation, they would be added as powdered salts (e.g., sulfate, chloride, or oxide) mixed into the gel coating of these soft-gel capsules. Depending upon the size of the individual, the therapeutic goal (prevention or treatment of BMS), and the degree of ongoing oxidative stress, the adult human dosage would range from 1-5 of these capsules daily.

The present invention also contemplates a test kit for assessing omega 3 and omega 6 statuses and BMS or risk thereof. The test kit comprises cotton gauze, brush or sponge type swab to get a cheek (buccal) cell sample, a sealable container, such as a test tube having a sealable top, in which the swab is placed and shipped to a testing facility and directions for use. The items may be contained in any suitable packaging material.

Once the subject receives the kit, he takes the swab and scrubs the inner surface of the cheek to take a sample of his buccal cells. The container is then at least partially filled with fluid, such as water. The swab having the buccal cells is then placed into the container. The container is then sealed. The cells are suspended in the fluid. The kit may also include an address to which the sample is to be sent for testing. After the sample is received by the testing facility, it is then subjected to fatty acid analysis using gas chromatography. This involves extracting fats from the cells using a non-polar solvent, hydrolysis, re-esterification to generate fatty acid methyl esters, and injection of an aliquot of this fatty acid methyl ester mix into a gas chromatograph that has been referenced with authentic fatty acid standards.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below. This completes the description of the preferred and alternate embodiments of the invention. Those skilled in the art may recognize other equivalents to the specific embodiment described herein which equivalents are intended to be encompassed by the claims attached hereto.

What is claimed is:

1. A method of treating a subject at risk for developing upper gastro-intestinal mucosal damage, comprising the steps of identifying the subject at risk for developing upper gastro-intestinal mucosal damage and replacing membrane arachidonic acid in the subject.

2. The method of claim 1, wherein the upper gastro-intestinal mucosal damage comprises gastric erosions, gastric ulcer(s) and/or duodenal ulcer(s).

3. The method of claim 1, wherein the upper gastro-intestinal mucosal damage is indicated by occult GI blood loss or by chronic low grade anemia.

4. The method of claim 1, wherein the subject is determined to be at risk for upper gastro-intestinal mucosal damage due to prolonged intense exercise.

5. The method of claim 1, wherein the subject is determined to be at risk for upper gastro-intestinal mucosal damage due to severe emotional stress.

6. The method of claim 1, wherein the subject is determined to be at risk for upper gastro-intestinal mucosal damage due to chronic infectious (septic) stress.

7. The method of claim 1, wherein the subject is determined to be at risk for upper gastro-intestinal mucosal damage due to smoking tobacco products.

8. The method of claim 1, wherein the subject is determined to be at risk for upper gastro-intestinal mucosal damage due to advanced age.

9. The method of claim 1, wherein the membrane arachidonic acid is replaced by feeding dietary arachidonic acid to the subject.

10. The method of claim 9, wherein the dietary arachidonic acid is fed in combination with a tocopherol.

11. The method of claim 10, wherein the tocopherol is chosen from the group consisting of gamma-tocopherol and delta-tocopherol.

12. The method of claim 9, where the dietary arachidonic acid is fed in combination with other nutrients mitigating ROS production or escape, such as omega-3 HUFA, copper, zinc, and selenium.

13. The method of claim 1, wherein the subject at risk for developing upper gastro-intestinal mucosal damage is identified due to a reduced membrane arachidonic acid level.

14. The method of claim 13, wherein the reduced membrane arachidonic acid is determined through analysis of cheek mucosal cells, gastric or small bowel mucosal cells, red or white blood cells, serum or plasma phospholipids, or serum or plasma total lipids or whole blood total lipid and or phospholipid

15. The method of claim 1, wherein the subject is a dog or other animal.

16. A method of treating a subject having upper gastro-intestinal mucosal damage, comprising the steps of determining if the subject has upper gastro-intestinal mucosal damage and replacing membrane arachidonic acid in the subject.

17. The method of claim 16, wherein the upper gastro-intestinal mucosal damage comprises gastric erosions, gastric ulcer(s) and/or duodenal ulcer(s).

18. The method of claim 16, wherein the gastro-intestinal mucosal damage is due to a reduction in membrane arachidonic acid.

19. The method of claim 18, wherein the reduction in membrane arachidonic acid is associated with prolonged intense exercise.

20. The method of claim 18, wherein the reduction in membrane arachidonic acid is associated with severe emotional stress.

21. The method of claim 18, wherein the reduction in membrane arachidonic acid is associated with chronic infectious (septic) stress.

22. The method of claim 18, wherein the reduction in membrane arachidonic acid is associated with the smoking of tobacco products.

23. The method of claim 16, wherein the membrane arachidonic acid is replaced by feeding dietary arachidonic acid to the subject.

24. The method of claim 23, wherein the dietary arachidonic acid is fed in combination with a tocopherol.

25. The method of claim 24, wherein the tocopherol is chosen from the group consisting of gamma-tocopherol, and delta-tocopherol.

26. The method of claim 18, wherein the reduced membrane arachidonic acid is determined through analysis of cheek mucosal cells, gastric or small bowel mucosal cells, red or white blood cells, serum or plasma phospholipids, or serum or plasma total lipids or whole blood total lipid or phospholipids

27. The method of claim 16, wherein the subject is a dog or other companion animal.

28. A quantity of food, wherein the food is specifically is designed for animal consumption and wherein the food includes dietary arachidonic acid for the specialized purpose of preventing or treating BMS and gastrointestinal bleeding.
